(19) United States
(12) Patent Application Publication
(10) Pub. No.: US 2023/0337922 A1
(43) Pub. Date: Oct. 26, 2023

(54) Device, System, and Method for Intraoperative Quantification of Blood Loss

(71) Applicant: H. Lee Moffitt Cancer Center and Research Institute, Inc., Tampa, FL (US)

(72) Inventor: Aaron Muncey, Tampa, FL (US)

(21) Appl. No.: 18/139,607

(22) Filed: Apr. 26, 2023

Related U.S. Application Data
(60) Provisional application No. 63/334,882, filed on Apr. 26, 2022.

(51) Int. Cl. A61B 5/02; A61M 60/216; A61B 5/00

(52) U.S. Cl. CPC A61B 5/02042; A61M 60/216; A61B 5/0075

(57) ABSTRACT

An interoperative blood loss quantification device that including a housing, a peristaltic pump positioned within the housing, a stepper motor positioned within the housing and driving the peristaltic pump, a cuvette positioned within the housing and configured to receive fluid moved by the peristaltic pump, a spectrophotometer positioned within the housing and configured to provide an absorbance signal indicative of properties of the fluid flowing through the cuvette, and one or more processing circuits that control operation of the stepper motor, determine a volume of fluid moved through the peristaltic pump, determine a blood concentration based on the absorbance signal received from the spectrophotometer, and determine a total volume of blood moved by the peristaltic pump based on the volume of fluid and the blood concentration

### CROSS REFERENCE TO RELATED APPLICATIONS

This application claims the benefit of U.S. Provisional Patent Application No. 63/334,882, filed on Apr. 26, 2022, the entire contents of which are incorporated herein by reference.

### BACKGROUND

This disclosure relates to blood loss quantification. More particularly, this disclosure relates to automated blood loss quantification in an operating room.

### SUMMARY

One embodiment relates to an interoperative blood loss quantification device that includes a housing, a peristaltic pump positioned within the housing, a stepper motor positioned within the housing and driving the peristaltic pump, a cuvette positioned within the housing and configured to receive fluid moved by the peristaltic pump, a spectrophotometer positioned within the housing and configured to provide an absorbance signal indicative of properties of the fluid flowing through the cuvette, and one or more processing circuits comprising one or more memory devices coupled to one or more processors, the one or more memory devices configured to store instructions thereon that, when executed by the one or more processors, cause the one or more processors to: control operation of the stepper motor, determine a volume of fluid moved through the peristaltic pump, determine a blood concentration based on the absorbance signal received from the spectrophotometer, and determine a total volume of blood moved by the peristaltic pump based on the volume of fluid and the blood concentration.

In some embodiments, the housing includes a vented body sized to contain the peristaltic pump, the stepper motor, the cuvette, the spectrophotometer, and the one or more processing circuits comprising one or more memory devices. The housing further includes a vented cap, a vented bottom bumper, and an operating room attachment bracket.

In some embodiments, the housing includes a caddy removably received within the housing and supporting the peristaltic pump, the stepper motor, the cuvette, the spectrophotometer, and the one or more processing circuits comprising one or more memory devices. In some embodiments, the caddy includes a first caddy configured to support the cuvette and the spectrophotometer, and a second caddie configured to support the peristaltic pump and the stepper motor.

In some embodiments, the interoperative blood loss quantification device further includes a display coupled to the housing, and the one or more memory devices are further configured to store instructions thereon that, when executed by the one or more processors, cause the one or more processors to generate a graphical user interface representing at least one of the volume of fluid moved through the peristaltic pump, the blood concentration, or the total volume of blood moved by the peristaltic pump.

In some embodiments, the interoperative blood loss quantification device further includes a communications interface configured to wirelessly transmit information from the interoperative blood loss quantification device including at least one of the volume of fluid moved through the peristaltic pump, the blood concentration, or the total volume of blood moved by the peristaltic pump.

In some embodiments, the spectrophotometer includes a laser configured to obtain a dual isosbestic point measurement.

In some embodiments, the spectrophotometer includes a first laser defining a first wavelength of about 520 nanometers, and a second laser defining a second wavelength of about 808 nanometers.

In some embodiments, the cuvette includes a cover configured to inhibit light pollution of the cuvette during use.

Another embodiment relates to an interoperative blood loss quantification system that includes a suction head, a main reservoir fluidly coupled to the suction head, a vacuum configured to provide a negative pressure within the main reservoir, a blood loss quantification device including: a peristaltic pump configured to move fluid from the main reservoir, a stepper motor driving the peristaltic pump, a cuvette configured to receive fluid moved by the peristaltic pump, a spectrophotometer configured to provide an absorbance signal indicative of properties of the fluid flowing through the cuvette, and one or more processing circuits comprising one or more memory devices coupled to one or more processors, the one or more memory devices configured to store instructions thereon that, when executed by the one or more processors, cause the one or more processors to: control operation of the stepper motor, determine a volume of fluid moved through the peristaltic pump, and determine a total volume of blood moved by the peristaltic pump based on the volume of fluid and the absorbance signal. The interoperative blood loss quantification system further includes an end reservoir fluidly connected to the blood loss quantification device.

In some embodiments, the interoperative blood loss quantification system further includes a safety reservoir positioned fluidly between the main reservoir and the vacuum.

In some embodiments, the one or more memory devices are further configured to store instructions thereon that, when executed by the one or more processors, cause the one or more processors to: determine a blood concentration based on the absorbance signal, and determine the total volume of blood moved by the peristaltic pump based on the volume of fluid and the blood concentration.

In some embodiments, the blood loss quantification device further includes a housing, and the peristaltic pump, the stepper motor, the cuvette, the spectrophotometer, and the one or more processing circuits comprising one or more memory devices are received within the housing.

In some embodiments, the blood loss quantification device further includes a housing and an operating room attachment bracket.

In some embodiments, the blood loss quantification device further includes a display, and the one or more memory devices are further configured to store instructions thereon that, when executed by the one or more processors, cause the one or more processors to generate a graphical user interface representing the total volume of blood moved by the peristaltic pump.

In some embodiments, the blood loss quantification device further includes a communications interface configured to wirelessly transmit information including at least one of the volume of fluid moved through the peristaltic pump, or the total volume of blood moved by the peristaltic pump.

In some embodiments, the cuvette includes a cover configured to inhibit light pollution of the cuvette during use, and the spectrophotometer includes a first laser defining a first wavelength of about 520 nanometers, and a second laser defining a second wavelength of about 808 nanometers.

Another embodiment relates to a method of interoperative blood loss quantification that includes moving a fluid from a main reservoir to a cuvette of a blood loss quantification device with a peristaltic pump, determining a blood concentration of the fluid, determining a total volume of fluid moved by the peristaltic pump, and determining a total blood volume moved by the peristaltic pump based on the blood concentration and the total volume of fluid.

In some embodiments, the method further includes determining the total blood volume based on a hemoglobin concentration.

In some embodiments, determining the blood concentration includes determining an absorbance value of the fluid using a spectrophotometer including a first laser defining a first wavelength of about 520 nanometers, and a second laser defining a second wavelength of about 808 nanometers.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### DESCRIPTION OF DRAWINGS

The device is explained in even greater detail in the following drawings. The drawings are merely exemplary and certain features may be used singularly or in combination with other features. The drawings are not necessarily drawn to scale.

FIG. 1 is a schematic diagram of an interoperative blood loss quantification system, according to some embodiments.

FIG. 2 is an exploded view of an interoperative blood loss quantification device of the interoperative blood loss quantification system of FIG. 1, according to some embodiments.

FIG. 3 is a perspective view of another interoperative blood loss quantification device, according to some embodiments.

FIG. 4 is an exploded view of the interoperative blood loss quantification device of FIG. 3, according to some embodiments.

FIG. 5 is a perspective view of a caddy of the interoperative blood loss quantification device, according to some embodiments.

FIG. 6 is a perspective view of a cuvette of the interoperative blood loss quantification device, according to some embodiments.

FIG. 7 is a schematic diagram of a controller of the interoperative blood loss quantification device, according to some embodiments.

FIG. 8 is a flow diagram of a method of operating the interoperative blood loss quantification device, according to some embodiments.

FIG. 9 is a perspective view of a top cover of the cuvette of FIG. 6, according to some embodiments.

FIG. 10 is a perspective view of a bottom cover of the cuvette of FIG. 6, according to some embodiments.

FIG. 11 is a front view of a cuvette housing of the cuvette of FIG. 6, according to some embodiments.

FIG. 12 is a perspective view of the cuvette housing of FIG. 11, according to some embodiments.

FIG. 13 is a perspective view of another cuvette housing, according to some embodiments.

FIG. 14 is a schematic diagram of a spectrophotometer circuit of the controller of FIG. 7, according to some embodiments.

FIG. 15 is a schematic diagram of a spectrophotometer circuit of the controller of FIG. 7, according to some embodiments.

FIG. 16 is a schematic diagram of a spectrophotometer circuit of the controller of FIG. 7, according to some embodiments.

### DETAILED DESCRIPTION

Following below are more detailed descriptions of concepts related to, and implementations of, methods, apparatuses, and systems for interoperative blood loss quantification. Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

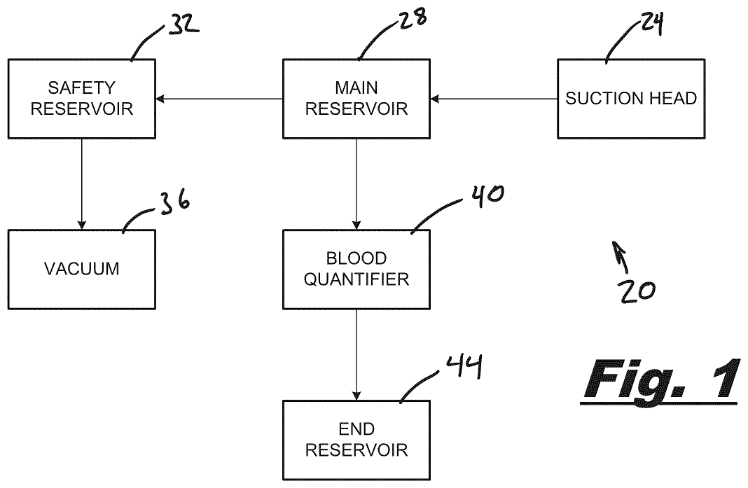

As shown in FIG. 1, an interoperative blood loss quantification system 20 includes a suction head 24 that removes fluid (e.g., a mixture of blood and other fluids) from a surgery site. The suction head is fluidly coupled to a main reservoir 28 that collects fluid sucked through the suction head 24. A safety reservoir 32 is fluidly coupled between the main reservoir 28 and a vacuum 36 to catch any fluid that may inadvertently escape the main reservoir 28 during suction. An interoperative blood loss quantification device 40 is arranged in fluid communication with the main reservoir 28 and can pull fluid therefrom. The blood loss quantification device 40 moves fluid from the main reservoir 28 to an end reservoir 44 and analyzes a total volume of fluid moved from the main reservoir 28 to the end reservoir 44, and a blood concentration of the moved fluid. Using the determined total volume and blood concentration, a total volume of blood loss is determined and can be used by a care team.

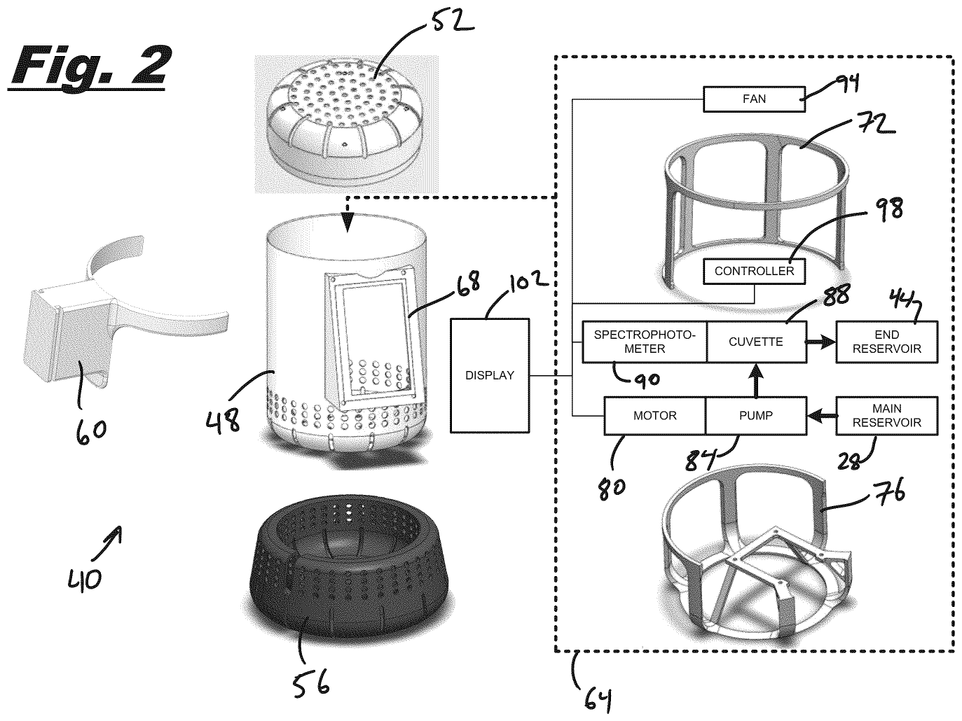

As shown in FIG. 2, the blood loss quantification device 40 includes a housing 48, a lid 52 covering an interior cavity of the housing 48, and a bottom bumper protecting a bottom portion of the housing 48. The housing 48, the lid 52, and the bumper 56 all include air flow apertures that provide air flow into and out of the housing 48 to control a temperature of components of the blood loss quantification device 40. An operating room attachment bracket 60 can be connected to the housing 48 to provide a mounting point for typical operating room canister holders. The housing 48 is sized to hold or encase blood quantifier components 64. The housing 48 also includes a display mount 68.

The blood quantifier components 64 include a caddy in the form of an upper caddy 72 and a lower caddy 76. A motor 80 and a peristaltic pump 84 are supported on the lower caddy 76. A cuvette 88 is arranged in fluid communication with the peristaltic pump 84 to receive fluid flow therefrom, and a spectrophotometer 90 is arranged to analyze fluid flowing through the cuvette 88. In some embodiments, the peristaltic pump 84 is arranged to draw fluid from the main reservoir 28 and push fluid through the cuvette 88 before the fluid is moved to the end reservoir 44. In some embodiments, the peristaltic pump 84 is positioned to draw the fluid from the main reservoir 28, through the cuvette 88, then through the peristaltic pump 84 before the fluid is pushed to the end reservoir 44. In some embodiments, the peristaltic pump 84 is a dual headed pump driven by the motor 80 arranged to both push fluid into the cuvette 88 and simultaneously pull the fluid out of the cuvette 88. In some embodiments, the motor 80 is a stepper motor. In some embodiments, the motor 80 is a servo motor.

In some embodiments, the cuvette 88, the spectrophotometer 90, and a controller 98 are supported by the upper caddy 72. The upper caddy 72 and the lower caddy 76 allow for easy installation and removal of the blood quantifier components 64 from the housing 48. For example, in some embodiments, all components that contact blood are replaced for each procedure including all piping. Easy access to the interior of the housing 48 facilitates this replacement.

A fan 94 is mounted to the lid 52 and provides forced air cooling to the motor 80 and the other blood quantifier components 64 during use.

The controller 98 controls operation of the blood quantifier components 64 as discussed in further detail below.

A display 102 (e.g., a human machine interface, a touch screen, etc.) is mounted to the display mount 68 of the housing 48 and provides information in real time to the user. The display also allows for users to input information to the blood quantifier components 64.

The interoperative blood loss quantification device 40 includes the housing 48, the peristaltic pump 84 positioned within the housing 48, the stepper motor 80 positioned within the housing 48 and driving the peristaltic pump 84, the cuvette 88 positioned within the housing 48 and configured to receive fluid moved by the peristaltic pump 84, the spectrophotometer 90 positioned within the housing 48 and configured to provide an absorbance signal indicative of properties of the fluid flowing through the cuvette 88, and one or more processing circuits 134 comprising one or more memory devices 142 coupled to one or more processors 138, the one or more memory devices 142 configured to store instructions thereon that, when executed by the one or more processors 138, cause the one or more processors to: control operation of the stepper motor 80, determine a volume of fluid moved through the peristaltic pump 84, determine a blood concentration based on the absorbance signal received from the spectrophotometer 90, and determine a total volume of blood moved by the peristaltic pump 84 based on the volume of fluid and the blood concentration.

In some embodiments, the housing 48 includes a vented body sized to contain the peristaltic pump 84, the stepper motor 80, the cuvette 88, the spectrophotometer 90, and the one or more processing circuits 134 comprising one or more memory devices 142. The housing 48 further includes a vented cap 52, a vented bottom bumper 56, and an operating room attachment bracket 60. In some embodiments, the housing 48 includes a vented body sized to contain the peristaltic pump 84, the stepper motor 80, the cuvette 88, the spectrophotometer 90, and the one or more processing circuits 134 comprising one or more memory devices 142. The housing 48 further includes a vented cap 52, a vented bottom bumper 56, and an operating room attachment bracket 60. In some embodiments, the housing 48 includes the caddy 72/76 removably received within the housing 48 and supporting the peristaltic pump 84, the stepper motor 80, the cuvette 88, the spectrophotometer 90, and the one or more processing circuits 134 comprising one or more memory devices 142. In some embodiments, the caddy 72/76 includes a first caddy 72 configured to support the cuvette 88 and the spectrophotometer 90, and a second caddie 76 configured to support the peristaltic pump 84 and the stepper motor 80. In some embodiments, the interoperative blood loss quantification device 40 further includes the display 102 coupled to the housing 48, and the one or more memory devices 142 are further configured to store instructions thereon that, when executed by the one or more processors 138, cause the one or more processors 138 to generate a graphical user interface representing at least one of the volume of fluid moved through the peristaltic pump 84, the blood concentration, or the total volume of blood moved by the peristaltic pump 84. In some embodiments, the interoperative blood loss quantification device 40 further includes a communications interface 168 configured to wirelessly transmit information from the interoperative blood loss quantification device 40 including at least one of the volume of fluid moved through the peristaltic pump 84, the blood concentration, or the total volume of blood moved by the peristaltic pump 84. In some embodiments, the spectrophotometer 90 includes a laser 118 configured to obtain a dual isosbestic point measurement. In some embodiments, the spectrophotometer 90 includes a first laser 118 defining a first wavelength of about 520 nanometers, and a second laser 118 defining a second wavelength of about 808 nanometers. In some embodiments, the cuvette 88 includes a cover 131 configured to inhibit light pollution of the cuvette 88 during use.

The interoperative blood loss quantification system includes the suction head 24, the main reservoir 28 fluidly coupled to the suction head 24, the vacuum 36 configured to provide a negative pressure within the main reservoir 28, the blood loss quantification device 40 including: the peristaltic pump 84 configured to move fluid from the main reservoir 28, the stepper motor 80 driving the peristaltic pump 84, the cuvette 88 configured to receive fluid moved by the peristaltic pump 84, the spectrophotometer 90 configured to provide an absorbance signal indicative of properties of the fluid flowing through the cuvette 88, and one or more processing circuits 134 comprising one or more memory devices 142 coupled to one or more processors 138, the one or more memory devices 142 configured to store instructions thereon that, when executed by the one or more processors 138, cause the one or more processors 138 to: control operation of the stepper motor 80, determine a volume of fluid moved through the peristaltic pump 84, and determine a total volume of blood moved by the peristaltic pump 84 based on the volume of fluid and the absorbance signal. The interoperative blood loss quantification system 20 further includes an end reservoir 44 fluidly connected to the blood loss quantification device 40.

In some embodiments, the interoperative blood loss quantification system 20 further includes the safety reservoir 32 positioned fluidly between the main reservoir 28 and the vacuum 36. In some embodiments, the one or more memory devices 142 are further configured to store instructions thereon that, when executed by the one or more processors 138, cause the one or more processors 138 to: determine a blood concentration based on the absorbance signal, and determine the total volume of blood moved by the peristaltic pump 84 based on the volume of fluid and the blood concentration. In some embodiments, the blood loss quantification device 40 further includes the housing 48, and the peristaltic pump 84, the stepper motor 80, the cuvette 88, the spectrophotometer 90, and the one or more processing circuits 134 comprising one or more memory devices 142 are received within the housing 48. In some embodiments, the blood loss quantification device 40 further includes the housing 48 and the operating room attachment bracket 60. In some embodiments, the blood loss quantification device 40 further includes the display 102, and the one or more memory devices 142 are further configured to store instructions thereon that, when executed by the one or more processors 138, cause the one or more processors 138 to generate a graphical user interface representing the total volume of blood moved by the peristaltic pump. In some embodiments, the blood loss quantification device 40 further includes the communications interface 168 configured to wirelessly transmit information including at least one of the volume of fluid moved through the peristaltic pump 84, or the total volume of blood moved by the peristaltic pump 84. In some embodiments, the cuvette 88 includes a cover 131 configured to inhibit light pollution of the cuvette during use, and the spectrophotometer 90 includes a first laser 118 defining a first wavelength of about 520 nanometers, and a second laser 118 defining a second wavelength of about 808 nanometers.

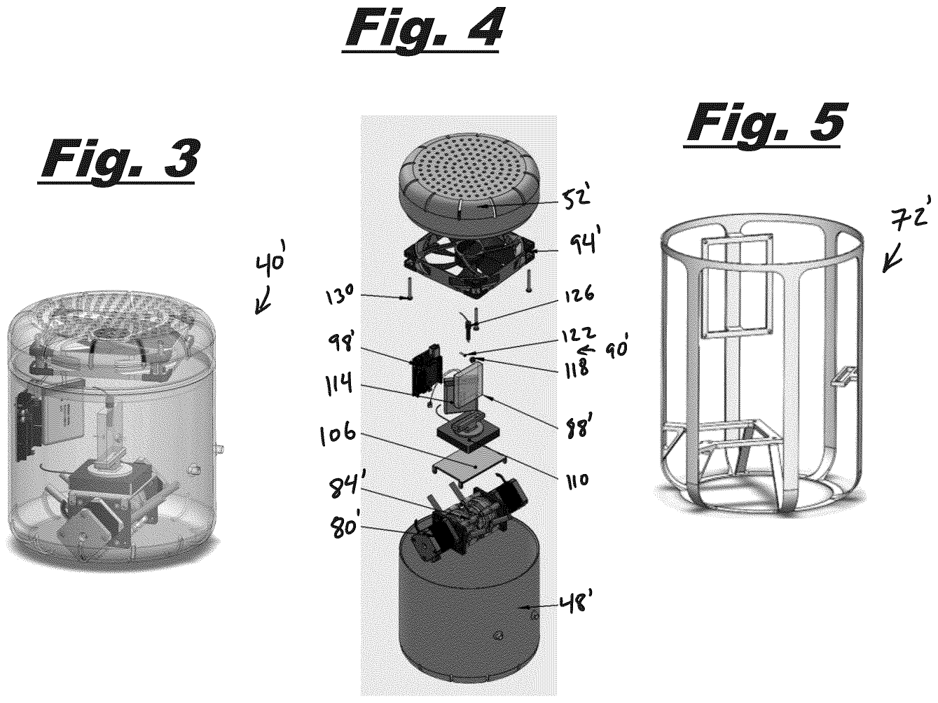

FIGS. 3-5 show another blood loss quantification device 40′ similar to the blood loss quantification device 40 discussed above with respect to FIGS. 1 and 2 and including similar components numbered in the prime series. The caddy 72′ is provided as a single piece caddy so that all blood quantifier components 64′ are removed together from the housing 48′. The blood loss quantification device 40′ further includes a plate 106, a scale 110 supported by the plate 106 above the peristaltic pump 84′, a battery 114 for providing power to the blood loss quantification device 40′ (e.g., a 2500 mAh battery), a laser diode 118, a photoresistor 122, and a conductivity sensor 126. The fan 94′ is fastened to the lid 52′ with screws 130.

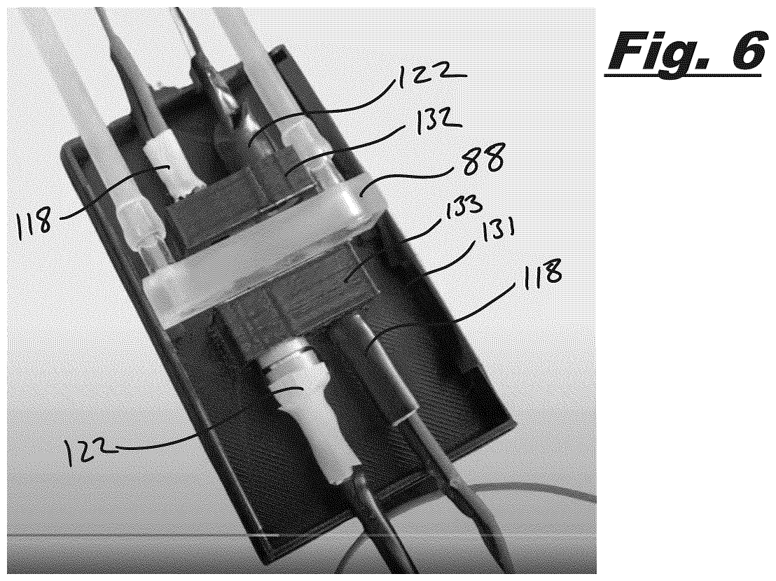

As shown in FIG. 6, the cuvette 88 is mounted in a cuvette housing 131 including a first mounting block 132 and a second mounting block 133. The cuvette housing 131 is constructed of opaque material to inhibit light pollution of the cuvette 88. The first mounting block 132 holds a first laser diode 118 that emits a first wavelength (e.g., 520 nanometers), and a second photoresistor that reacts to a second wavelength (e.g., 808 nanometers). The second mounting block 133 holds a second laser diode 118 that emits the second wavelength, and a first photoresistor that reacts to the first wavelength. The first laser diode 118 is aligned with the first photoresistor through the cuvette 88, and the second laser diode 118 is aligned with the second photoresistor through the cuvette 88.

The cuvette 88 is constructed of a clear material such as quartz and defines an inlet, and outlet, and a flow chamber through which fluid flows. The cuvette is shaped to provide even flow through the flow chamber and inhibit the coagulation of blood and other fluids during use. In some embodiments, the peristaltic pump 84 is cycled backward and forward during times of low or no flow to further inhibit coagulation within the cuvette 88. In some embodiments, only one set of laser diode and photoresistor are provided with the spectrophotometer 90. In some embodiments, more than two sets of laser diodes and photoresistors are provided with the spectrophotometer 90 to provide additional absorption information allowing the care team to better understand the fluids exiting the patient.

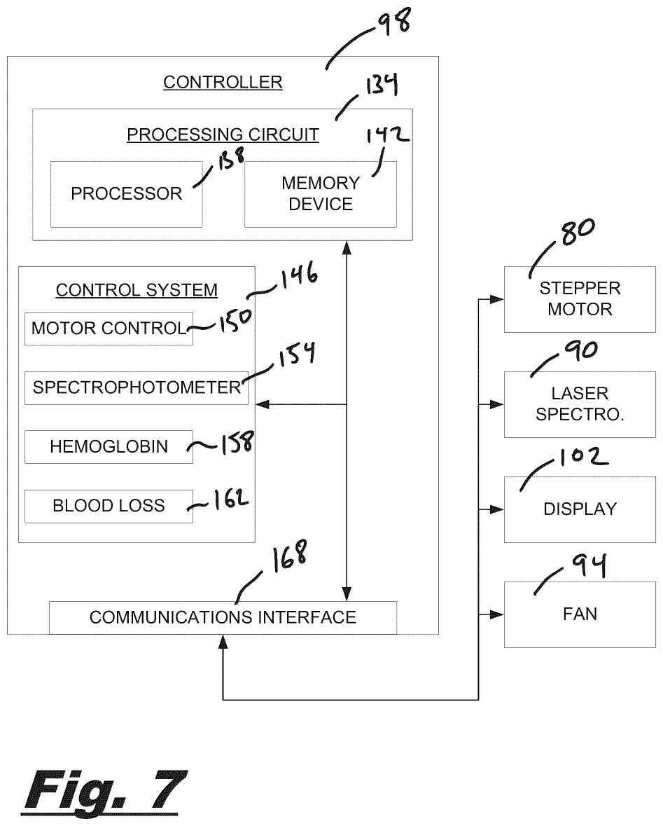

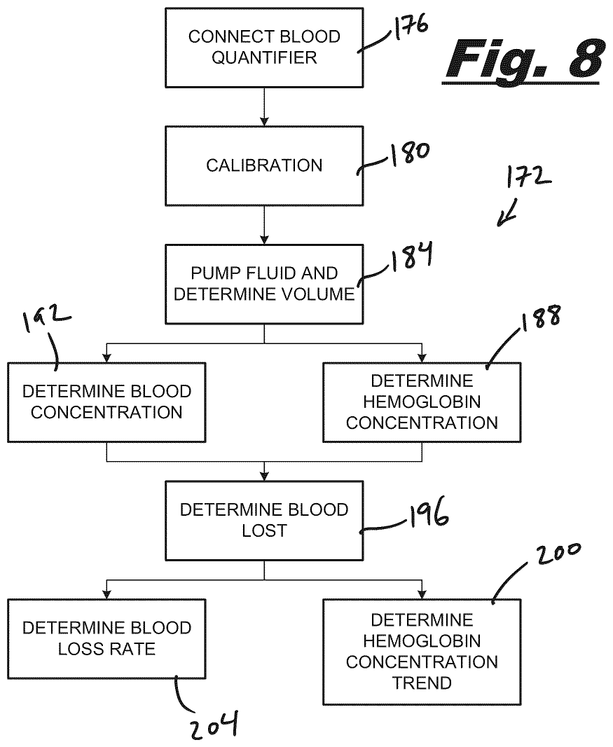

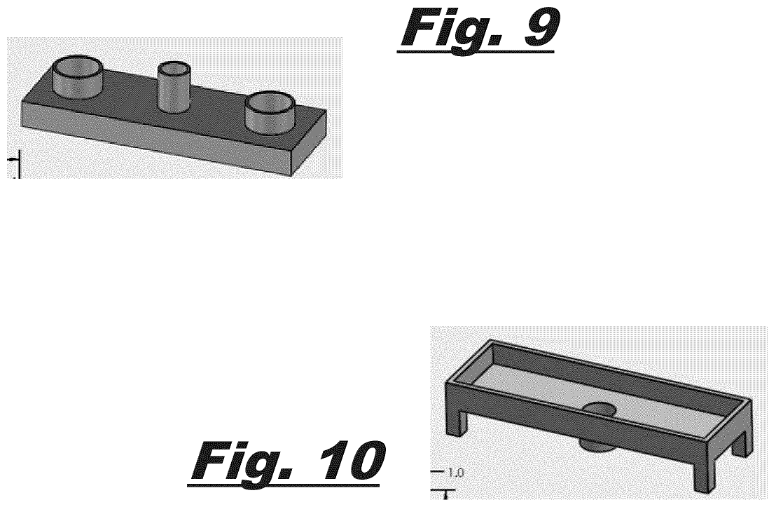

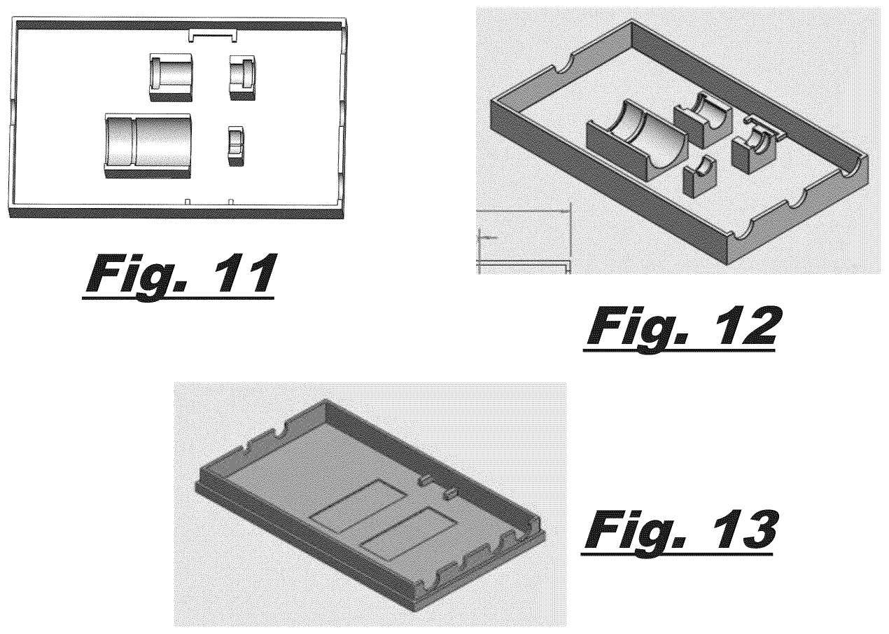

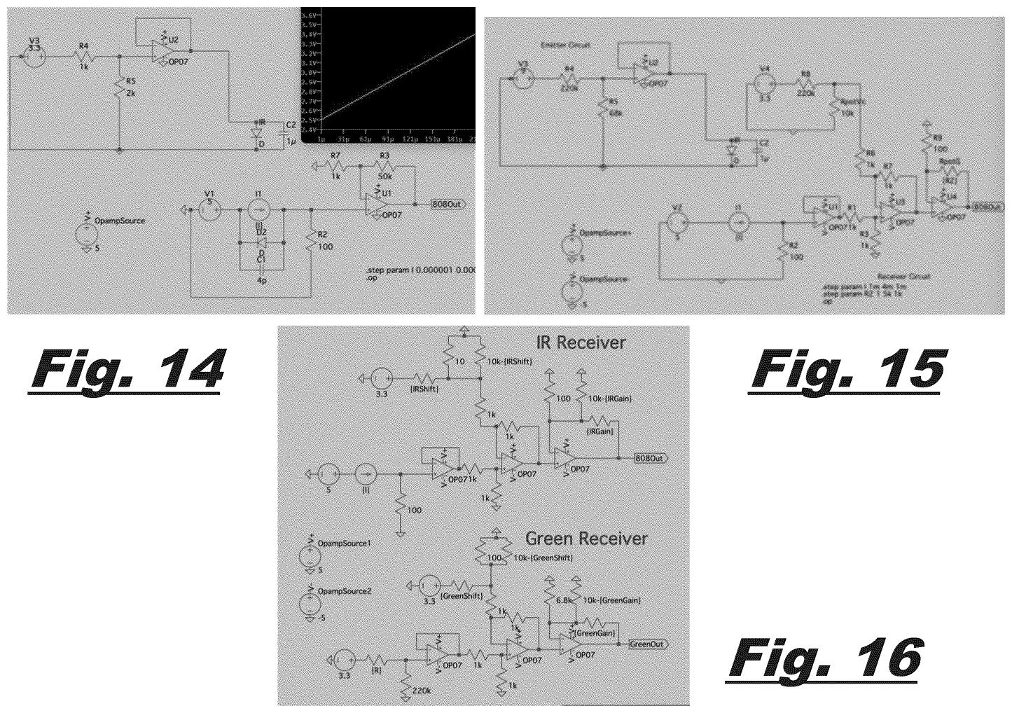

Referring now to FIG. 7, a schematic diagram of the controller 98 is shown according to an example embodiment. As shown in FIG. 7, the controller 98 includes a processing circuit 134 having a processor 138 and a memory device 142, a control system 146 having a motor control circuit 150, a spectrophotometer circuit 154, a hemoglobin circuit 158, and a blood loss circuit 162, and a communications interface 168. Generally, the controller 98 is structured to operate the motor 80 to move a quantified volume of fluid through the peristaltic pump 84, operate the spectrophotometer 90 to determine a blood concentration of the fluid moving through the cuvette 88, determine a hemoglobin level of the patient, and determine a total blood loss based on the hemoglobin level, the blood concentration, and the total volume of fluid. The controller 98 also generates a graphical user interface (GUI) that is provided to the user via the display 102. The controller 98 also communicates with hospital systems either wirelessly (e.g., via Bluetooth®) or via a wired connection to provide an integrated interoperative blood loss quantification system 20.

In one configuration, the circuits of the control system 146 are embodied as machine or computer-readable media that is executable by a processor, such as processor 138. As described herein and amongst other uses, the machine-readable media facilitates performance of certain operations to enable reception and transmission of data. For example, the machine-readable media may provide an instruction (e.g., command, etc.) to, e.g., acquire data. In this regard, the machine-readable media may include programmable logic that defines the frequency of acquisition of the data (or, transmission of the data). The computer readable media may include code, which may be written in any programming language including, but not limited to, Java or the like and any conventional procedural programming languages, such as the “C” programming language or similar programming languages. The computer readable program code may be executed on one processor or multiple remote processors. In the latter scenario, the remote processors may be connected to each other through any type of network (e.g., CAN bus, etc.).

In another configuration, the circuits of the control system 146 are embodied as hardware units, such as electronic control units. As such, the circuits of the control system 146 may be embodied as one or more circuitry components including, but not limited to, processing circuitry, network interfaces, peripheral devices, input devices, output devices, sensors, etc. In some embodiments, the circuits of the control system 146 may take the form of one or more analog circuits, electronic circuits (e.g., integrated circuits (IC), discrete circuits, system on a chip (SOCs) circuits, microcontrollers, etc.), telecommunication circuits, hybrid circuits, and any other type of “circuit.” In this regard, the circuits of the control system 146 may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so on). The circuits of the control system 146 may also include programmable hardware devices such as field programmable gate arrays, programmable array logic, programmable logic devices or the like. The circuits of the control system 146 may include one or more memory devices for storing instructions that are executable by the processor(s) of the circuits of the control system 146. The one or more memory devices and processor(s) may have the same definition as provided below with respect to the memory device 142 and processor 138. In some hardware unit configurations, the circuits of the control system 146 may be geographically dispersed throughout separate locations in the power system. Alternatively and as shown, the circuits of the control system 146 may be embodied in or within a single unit/housing, which is shown as the controller 98.

In the example shown, the controller 98 includes the processing circuit 134 having the processor 138 and the memory device 142. The processing circuit 134 may be structured or configured to execute or implement the instructions, commands, and/or control processes described herein with respect to the circuits of the control system 146. The depicted configuration represents the circuits of the control system 146 as machine or computer-readable media. However, as mentioned above, this illustration is not meant to be limiting as the present disclosure contemplates other embodiments where the circuits of the control system 146, or at least one circuit of the circuits of the control system 146, is configured as a hardware unit. All such combinations and variations are intended to fall within the scope of the present disclosure.

The hardware and data processing components used to implement the various processes, operations, illustrative logics, logical blocks, modules and circuits described in connection with the embodiments disclosed herein (e.g., the processor 138) may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the one or more processors may be shared by multiple circuits (e.g., the circuits of the control system 146 may comprise or otherwise share the same processor which, in some example embodiments, may execute instructions stored, or otherwise accessed, via different areas of memory). Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. All such variations are intended to fall within the scope of the present disclosure.

The memory device 142 (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory device 142 may be communicably connected to the processor 138 to provide computer code or instructions to the processor 138 for executing at least some of the processes described herein. Moreover, the memory device 142 may be or include tangible, non-transient volatile memory or non-volatile memory. Accordingly, the memory device 142 may include database components, object code components, script components, or any other type of information structure for supporting the various activities and information structures described herein.

The motor control circuit 150 is structured to control operation of the motor 80 to drive the peristaltic pump 84 and move fluid from the main reservoir 28, through the cuvette 88, and to the end reservoir 44. In some embodiments, the motor control circuit 150 receives sensor information indicative of a fluid level in the main reservoir 28 and operates the pump to maintain a fluid level in the main reservoir 28 below a threshold volume. In some embodiments, the motor control circuit 150 also calculates a total volume of fluid moved from the main reservoir 28.

The spectrophotometer circuit 154 is structured to operate the spectrophotometer 90 and determine an absorbance value of the fluid using the laser diodes 118 and photoresistors 122 at the isosbestic points of hemoglobin.

The hemoglobin circuit 158 is structured to receive the absorbance value from the spectrophotometer circuit 154 and determine a hemoglobin concentration and a blood concentration of the fluid. The hemoglobin circuit 158 also determines a hemoglobin concentration rate over time and a blood concentration rate over time.

The blood loss circuit 162 is structured to determine a total blood loss volume based on the total volume of fluid determined by the motor control circuit 150 and the blood concentration determined by the hemoglobin circuit 158.

The communications interface 168 can provide wireless communications (e.g., Bluetooth®) to external systems, and also communicate with the quantifier components 64 of the blood loss quantifier device 40 (e.g., the motor 80, the spectrophotometer 90, the display 102, and the fan 94).

While various circuits with particular functionality are shown in FIG. 7, it should be understood that the controller 98 may include any number of circuits for completing the functions described herein. For example, the activities and functionalities of the circuits of the control system 146 may be combined in multiple circuits or as a single circuit. Additional circuits with additional functionality may also be included. Further, the controller 98 may further control other activity beyond the scope of the present disclosure. In some embodiments, the circuits described herein may include one or more processing circuits comprising one or more memory devices coupled to one or more processors, the one or more memory devices configured to store instructions thereon that, when executed by the one or more processors, cause the one or more processors to perform the operations performed herein and described with reference to circuits.

As mentioned above and in one configuration, the “circuits” may be implemented in machine-readable medium for execution by various types of processors, such as the processor 138 of FIG. 7. An identified circuit of executable code may, for instance, comprise one or more physical or logical blocks of computer instructions, which may, for instance, be organized as an object, procedure, or function. Nevertheless, the executables of an identified circuit need not be physically located together, but may comprise disparate instructions stored in different locations which, when joined logically together, comprise the circuit and achieve the stated purpose for the circuit. Indeed, a circuit of computer readable program code may be a single instruction, or many instructions, and may even be distributed over several different code segments, among different programs, and across several memory devices. Similarly, operational data may be identified and illustrated herein within circuits, and may be embodied in any suitable form and organized within any suitable type of data structure. The operational data may be collected as a single data set, or may be distributed over different locations including over different storage devices, and may exist, at least partially, merely as electronic signals on a system or network.

While the term “processor” is briefly defined above, the term “processor” and “processing circuit” are meant to be broadly interpreted. In this regard and as mentioned above, the “processor” may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given circuit or components thereof may be disposed locally (e.g., as part of a local server, a local computing system, etc.) or remotely (e.g., as part of a remote server such as a cloud based server). To that end, a “circuit” as described herein may include components that are distributed across one or more locations.

Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

As shown in FIG. 8, a method 172 of interoperative blood loss quantification includes moving a fluid from a main reservoir to a cuvette of a blood loss quantification device with a peristaltic pump, determining a blood concentration of the fluid, determining a total volume of fluid moved by the peristaltic pump, and determining a total blood volume moved by the peristaltic pump based on the blood concentration and the total volume of fluid. In some embodiments, the method also includes determining the total blood volume based on a hemoglobin concentration. In some embodiments, determining the blood concentration includes determining an absorbance value of the fluid using a spectrophotometer including a first laser defining a first wavelength of about 520 nanometers, and a second laser defining a second wavelength of about 808 nanometers.

At step 176, the blood loss quantification device 40 is installed into the blood loss quantification system 20. At step 180, the blood loss quantification device 40 is calibrated to the patients blood. This may be accomplished using a pre-drawn sample of fluid that has been independently tested and verified so that a hemoglobin concentration and blood concentration are known. The blood loss quantification device 40 then calibrates the spectrophotometer circuit 154 to the patient's fluids.

At step 184, the motor 80 is operated so that the fluid is moved by the pump 84 from the main reservoir 28, through the cuvette 88, and into the end reservoir 44. The motor control circuit 150 determines the total volume of fluid as the pump 84 is operated.

At step 188 the hemoglobin circuit 158 determines the hemoglobin concentration based on the information received from the spectrophotometer circuit 154. At step 192 the hemoglobin circuit 158 determines the blood concentration based on the information received from the spectrophotometer circuit 154.

At step 196, the blood loss circuit 162 determines a total volume of blood lost based on the blood concentration, the hemoglobin concentration, and the total fluid volume. At step 200, the hemoglobin circuit 158 determines a hemoglobin concentration rate over time allowing the care team to monitor changes over time and absolute concentration levels in real time. At step 204, the hemoglobin circuit 158 determines a blood concentration rate over time allowing the care team to monitor changes over time and absolute concentration levels in real time.

Exemplary device capabilities are listed below. Adaptive to OR technology: the device was designed to be operated alongside the current blood canister setup present in Operation Rooms. The input of the device comes from one of the blood canisters and the output is to a second canister. The device sits on the rack that holds up to four canisters but takes the place of one of them. Automated blood loss monitoring: the entire operation of the device is fully autonomous besides an initial calibration that allows for the device to be patient-specific and provide more accurate data. Quantify the total volume of fluid suctioned to the canisters: the device moves the fluid using a peristaltic pump with a stepper motor. The steps of the motor are related to the volume pumped through the valve. Quantify blood concentration on blood canisters: using a two-laser system at the isosbestic points of hemoglobin, the device can calculate the absorbance values of the fluid flowing through a flow-through quartz cuvette. These absorbance values are directly correlated to the concentration of patient blood in the suctioned solution. Estimation of blood loss given patient's Hb level: given the patient's hemoglobin concentration, the device estimates the amount of blood lost during the surgery by using the Hb concentration data and the total volume data. Therefore the device is able to separate blood from other fluids such as saline that are present in the blood canisters. Calculation of the rate of fluid input to canisters: as the device tracks the fluid input over time, a rate calculation can be easily performed. Calculation of the rate of Hb concentration: since the device tracks the Hb concentration over time, a rate calculation can be easily performed. Estimation of the rate of blood loss over time: the device is capable of tracking the total blood loss in real time by assuming a constant Hb concentration inside the patient. The device uses the total fluid volume reading and the Hb absorbance data to calculate the Hb concentration in the total fluid suctioned in the canister. If it receives data from the patient's Hb concentration it can then calculate how much of the total fluid in the canisters is blood. It does that by comparing and matching the Hb concentrations of the total fluid and the patient's blood. Technical aspects: dual isosbestic point measurement with lasers (520 nm and 808 nm), flow through cuvette (quartz), stepper motor step for volume calculation, shape and size to fit canisters rack, and Bluetooth® enabled to transmit data.

One goal was to produce a method or device that improved patient outcomes after any surgery by assisting in determining the necessity of a blood transfusion. However, the strategic focus was first placed on “major surgeries that pose a high risk of blood loss and potentially require transfusion” and then broadened to generalize the solution for less invasive surgeries that required transfusions less frequently.

Major blood loss, or hypovolemia, is defined as a loss of at least 20% of a patient's total blood volume. The surgeries most often associated with severe bleeding are cardiovascular procedures, liver transplantation and hepatic resections, and major orthopedic procedures including hip and knee replacement and spine surgery. If blood loss approaches the 30-40% of the total blood volume lost, the patient is deemed critical and blood transfusions are necessary. Blood loss has major implications for lack of oxygenation of tissue and maintaining oncotic pressure for perfusion. Therefore, the primary causes of required blood transfusions are these surgeries, though other procedures exist that result in major blood loss. Further, this project targets individuals requiring these major surgeries, as they are the most likely population to undergo blood loss and require transfusions.

Blood transfusions were shown to be associated with a significant decrease in the post-operative survival time of patients who required curative resection of lung carcinoma. This finding supported the hypothesis that blood transfusions are responsible for a substandard prognosis in patients who undergo resection for carcinoma of the lung. This operation directly relates to Moffitt's strategic focus, as the lungs are reported to be the fourth most frequent site of cancer (8.5% of cases). Therefore, blood transfusions pose significant risk to patient health.

Current methods for monitoring the quantity of blood lost during surgeries are limited in their accuracy. Visual estimation is used consistently, though the accuracy of this technique is very poor. It has been reported that personnel miscalculated volume of blood lost by a median value of 30% regardless of profession, years of experience, and self-assessed ability about visual estimation. It has been suggested that spectrophotometric analysis of hemoglobin concentration can accurately assess blood loss. However, this process takes too long to perform in the operation room, and so gravimetric evaluation, while significantly less accurate, is the recommended approach for clinical settings.

One common side-effect of blood transfusion is depression of the patient's immune system. This is a particular issue for cancer patients, as their already deficient immune systems leave them vulnerable to developing severe symptoms from mild illnesses. It is reported that liver resection is widely accepted as the standard treatment for individuals suffering from benign and malignant tumors in the liver. Thus, the standard treatment for liver cancer has the capacity to damage the immune system of the patients, who undoubtedly already are immunocompromised. Further, it is currently believed that red blood cell and platelet transfusions contribute to the risk of post-operative venous and arterial thromboembolism in patients with cancer. Thus, those requiring invasive surgeries are likely to take on these increased risks. While blood transfusions are clearly preferable to the alternative of major blood loss leading to death, unnecessary blood transfusions pose unnecessary risks.

A more efficient protocol and medical practice involving blood transfusions is necessary to avoid a future shortage of blood supply and increase the value of health care. Dr. Goodnough conducted research on coronary artery bypass graft surgery in 1993 where his findings demonstrated that 27% of all blood units transfused across 18 institutions were unnecessary, and that 47%, 32%, and 15% of all platelets, plasma, and red cell units, respectively, were transfused inappropriately. Although this article is nearly 30 years old, a newer article from 2011 had 15 experts reviewing 494 published articles and concluded that allogenic red blood transfusion was inappropriate in 59.3% of them. This reveals that blood transfusion practice is still highly inefficient.

With an average charge to the patient of $343 per unit of blood (˜500 mL) and a total cost of $3,671.67 for the service (based on The Johns Hopkins Hospital pricing), it is evident that transfusion optimization provides tangible benefits for the healthcare industry. Knowing that approximately 50% of the transfusions are unnecessary or inefficient, one can see how the cost of healthcare is severely impacted by the lack of a process that accurately dictates the need for blood transfusion. It is known that hematology and oncology, together with gastroenterology general surgery, emergency medicine, cardiothoracic and vascular surgery were responsible for over 50% of the blood transfusions in 2010. Therefore, a way to accurately measure the need for blood transfusion can have a major impact on not only the cost of the healthcare system but improved clinical patient outcomes.

Spectrophotometric hemoglobin (SpHb) monitoring is a noninvasive way to measure hemoglobin concentration in blood and therefore track if the patient is becoming anemic. This method is being implemented on surgeries with high risk of blood loss. This technology provides a real time reading of hemoglobin levels, which are a good indicative of the need of red blood cell transfusion. SpHb works by emitting multiple wavelengths and then calculates hemoglobin concentration based on light absorption.

Blood loss is defined as either external or internal, and severity depends on volume lost and how rapidly it is lost. A range of under 15% blood lost is generally negligible, but above results in mean arterial/pulse pressure drops and heart rate jumps. More than 40% is life threatening. Firstly, blood loss causes a decrease in pressure, causing sympathetic response to systemically vasoconstrict certain organ vessels, directing blood to the brain and myocardium. This results in acidosis for those organs as waste products can't be perfused out of the organs. Further pressure drops lead to hypotension, and when pressure is typically the driving force for capillary movement of fluids, fluid goes back into the capillary from tissues, which includes proteins and electrolytes, diluting the blood and decreasing the hematocrit. Even if blood volume is brought to normal levels, if pressure isn't, myocardial hypoxia and acidosis occur, and tissues won't be able to oxidize electron carriers in the mitochondria that provides the ATP required for cellular function. It's also important to note that blood circulation is important for temperature modulation.

Several diseases that require invasive surgeries can result in blood loss. Most oncological procedures that result in removal of tumors can result in such, thrombolysis procedures, heart valve replacements, transplants etc. Particular areas of notice are obstetrics, cardiovascular, and urinary procedures, due to their high association with blood. For internal based diseases and blood loss, liver disease, gastritis, hemophilia, Von Willebrand's disease, and other conditions can result in internal bleeding.

Hypovolemia has four main stages. First is a loss of 750 cc, or up to 15%, with narrowing blood vessels to maintain pressure, although heart rate is normal. Urine production occurs normally. Above this loss to 1500 cc results in a rising heart rate, vasoconstriction of lesser organs. Above 1500 cc causes drops of blood pressure, slowing urine production, and more than 2000 cc drops urine production entirely, and a large drop of blood pressure. The onset of the symptoms mentioned above occurs, and death is likely. There are obvious exceptions, due to patient in question, different injuries, age, comorbidities, medication, so on; bradycardia (slow heart rate) can also occur.

Medical equipment is usually disposable, specifically tubing, and plastic equipment. The devices discussed above include peristaltic pumps, where the tubing can be disposable and nothing else would touch the blood.

The above devices can be broken down for understanding by looking at each of its internal components and the process by which the blood travels to the device. The suction wand that the institute uses feeds into the standard reservoir canister, which has two outlet pathways connected via medical grade tubing. One pathway is connected to an overflow canister with a vacuum to provide the pressure gradient that allows fluid to be suctioned into the system. This means that if the blood volume within the reservoir gets too high, the vacuum would allow fluid to flow from the reservoir to the overflow canister to prevent any backflow of fluid back into the patient via the suction wand. The other pathway leads to the novel component of the system, the sensory canister. The sensory canister is a blacked-out canister that houses the sensing equipment (spectrophotometer), circuitry and microcontroller components, peristaltic pumps, an integrated scale, batteries, and a fan and perforation for venting. A peristaltic pump is implemented internally within the sensory canister and is applied to the tubing that connects the reservoir to the sensor. The pump is controlled by a microcontroller and a stepper motor. This tubing leads to the bottom of an open top cuvette with two parallel sides blacked out.

Spectrophotometry relies heavily on the Beer-Lambert Law. The Beer-Lamber Law is a linear relationship correlating light absorbance to concentration of a particular solution. Implementation of this relationship allows for the use of light and its absorbance in a particular sample to determine the concentration of that solution based on developed calibration curves. This is the approach that the team currently plans on pursuing by utilizing laser emitters and receivers to determine absorbance in blood solutions diluted with saline and potentially other bodily fluids.

The ISO 6710:2017 is the standard for collection of blood in single use containers. This standard was considered for the fact that blood will be entering the device via disposable tubes and into a disposable cuvette. In this case the device will act as a single use container for the blood during analysis as these components are to be replaced after each surgery.

The ISO 14847:1999: is the standard for technical requirements for Rotary Positive Displacement Pumps which will be addressed with the use of a market available peristaltic pump already fabricated/cleared and will be purchased in the future. This standard includes details related to the specifications of the pump itself, which play a role in the packaging of the product. Use of this standard also implements how the pump deals with various fluids, such as blood, and its ability to maintain constant pumping ability despite discrete components in the fluid.

The IEC 60601-1:2005+AMD1:2012+AMD2:2020 is the overarching standard for basic safety and functional performance of medical electrical equipment, and the tests associated with them. This standard is for use in both professional healthcare facilities as well as home healthcare environments, for which the device will have to meet as it is a point of care device. This standard was considered due to its use of electronic controls for managing blood flow throughout the device.

The ISO 13732-1:2006(en) (part 1) is a standard regarding a hot environment and the methods with which humans should interact with hot surfaces. This was important when considering the device casing as it could overheat due to the motors encased inside. The fan was included to help prevent or deal with this issue; however, it is likely that the surface of the device might still become hot (See also IEC 60601-1, IEC 60950-1 and IEC 61010-1AN-G012 for more).

The ISO/IEC 17025 Testing and Calibration Laboratories is a standard that comes into play for testing the device for an accurate spectrophotometer and mass balance reading. The analysis will need to be compared against a calibration curve obtained from testing blood samples as well as weight values, respectively, during fabrication of the device so it can perform accurately once it reaches the market.

The ISO 13485:2016 Medical Devices is the overarching standard for quality management and played a crucial role in the proposed medical device. Quality is divided into policy, objectives, and manual, and these components make up the Quality Management System for an organization to produce a product. This includes standards for risk associated with the device, responsibilities for use, and handling. The standard is meant to assure consumers that the product is a verifiable product that performs consistently, especially in the context of real time blood analysis where surgical staff depend on accurate information for informed decisions.

The ISO 14971 Medical Devices is the overarching standard for the processes of risk management of a medical device. Meeting these standards means identifying the hazards associated with the device, its biocompatibility, electricity, moving parts, and usability and how well these risks are controlled for within the device. These risks are documented such that they play a role in the life cycle of a medical device and ensure whether the device for real time blood analysis will be robust for consumer purposes.

In some embodiments, the device was not meant to be interacted with, but it was intended to feel like a plastic cylinder. For all the inner components to fit in the device it needed to have a diameter of 20 cm and a height of 23 cm, a reasonable size to fit in the OR. Furthermore, the device utilizes the existing blood canisters as blood containers, and the device is compatible with the tubing the OR staff uses, therefore, allowing easy integration with the existing medical equipment.

Briefly, the functional circuit used an 808 nm infrared laser and a photodiode. The samples were placed between the photodiode and the laser, where the more concentrated the blood, the lower the voltage reading was. In some embodiments, the pump, the spectrophotometry circuit, and all working parts of the device are controlled by a MSP430FR2355 microcontroller.

The LTSpice model of this circuit is also presented in FIG. 14. This circuit utilizes an infrared (e.g., 808 nm wavelength) emitter and an infrared receiver photodiode to detect the intensity of infrared light passing through the cuvette holder (square hole in gray fixture). IR light contact with the photodiode produced a small current proportional to the light intensity that then induced a voltage drop across a 100 Ohm resistor. This drop is then amplified by a gain of 69 and read by an oscilloscope. This allows for the creation of calibration curves relating blood concentration of voltage output.

Implementation of the peristaltic pump provides a method of moving patient blood/fluid without expensive components directly contacting the fluid. Common practice dictates that components that touch bodily fluids were to be discarded after surgery. The use of the peristaltic pump may eliminate the need for valves and limited the components that touched the blood to only the tubing, cuvette, and any relevant connector pieces. It may be possible to utilize cleaning fluids to avoid disposal of the cuvette.

A COMSOL simulation for blood flow through the cuvette was conducted in meters per second. A Newtonian, stationary study was applied to achieve the results; this simulation was applied to determine if the fluid would be homogenous at the center of the cuvette to represent most of the fluid passing through the cuvette, allowing for the laser to sample the fluid and represent the solution. From both the top-down view and the side view, the fluid was homogenous at the center, meaning the computational estimations via the laser were representative of the fluid, although there were several considerations to make. First, blood is a non-Newtonian fluid with dynamic properties that depend on the situation, and this behavior was not modelled here. Second, a time dependent study which simulates the sporadic suctioning of the blood and models in real time would demonstrate whether blood flow was homogenous throughout the cuvette.

This modeling was performed to locate any points within the cuvette where blood would stagnate. It is necessary for both lasers to pass through the cuvette at a location that was consistently supplied with new blood. This was because processing stagnant blood results in inaccurate readings and potential coagulation. Thus, the results of this model verified the planned laser placement and led to further consideration of occasionally oscillating the peristaltic pump to perturb any stagnation.

The cuvette case design shown in FIGS. 11 and 12 positions all the sensing components in a box that keeps the environment surrounding the cuvette dark and reduces outside interference from light. The case is produced from ASA, the same material as the rest of the structure.

The caddy insert component shown in FIG. 5 provides a ledge type of support to hold the cuvette box in place on the perimeter of the caddy.

The cuvette box shown in FIG. 13 is slightly altered relative to the cuvette box shown in FIGS. 11 and 12. The laser/receivers are flush with the surface of the cuvette rather than spaced apart from it. The square cut out spots are where the laser box is glued to hold the laser in place. The cutout is flush with the slot where the cuvette sits.

In some embodiments, different wavelengths are used that that would allow for measurements and cross checking of arterial versus venous blood, which may be important to surgical staff concerning patient health in perioperative settings. In some embodiments, an SD card slot for the display is accessible once the display is placed within the device, such that the display doesn't have to be removed each time a relevant change was made. In some embodiments, solutions that inhibit the adaptation of the IR diode, such as ventilation of the cuvette case without any light entering, allow for more precise readings.

Given the desire to calibrate the device to patient-specific blood, the ability to fit calibration curves to adjustable endpoints is advantageous. It can be reasonably assumed that the voltage readings for 0% blood will be consistent; however, the voltage readings corresponding to 100% patient blood will almost certainly change depending upon the patient's starting hematocrit.

Stretching of the calibration curve itself was accomplished through the introduction of two variables: one which shifts the curve along the x-axis (voltage) and one which shifts the curve along the y-axis (concentration). As the infrared receiver response is just a line, adjustment is extraordinarily easy. However, the green receiver response is significantly more complicated, and so the necessary math was more sophisticated. A concentration vs. voltage equation for the green circuit with variables y (x-shift) and x (y-shift) allow for the user to record the voltage at 100% blood solution, save this value as “Vmin,” record the voltage at 0% blood solution, save this value as “Vmax,” and then calibrate the system. In some embodiments, preoperative calibration is accomplished by scaling of the y-axis (concentration) only. Essentially, the user would input 100% patient blood, and the device would output the estimated concentration, which will be called C for now. The device would then create a gain of 100/C and multiply the response by this gain, thus scaling the y-axis.

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

Features disclosed in this specification (including any accompanying claims, abstract, and drawings), and/or all of the steps of any method or process so disclosed, may be combined in any combination, except combinations where at least some of such features and/or steps are mutually exclusive. The claimed features extend to any novel one, or any novel combination, of the features disclosed in this specification (including any accompanying claims, abstract, and drawings), or to any novel one, or any novel combination, of the steps of any method or process so disclosed.

As used in the specification and the appended claims, the singular forms “a”, “an”, and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

The terms “coupled”, “connected”, and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic. For example, circuit A communicably “coupled” to circuit B may signify that the circuit A communicates directly with circuit B (i.e., no intermediary) or communicates indirectly with circuit B (e.g., through one or more intermediaries).

Certain terminology is used in the following description for convenience only and is not limiting. The words “right”, “left”, “lower”, and “upper” designate direction in the drawings to which reference is made. The words “inner” and “outer” refer to directions toward and away from, respectively, the geometric center of the described feature or device. The words “distal” and “proximal” refer to directions taken in context of the item described and, with regard to the instruments herein described, are typically based on the perspective of the practitioner using such instrument, with “proximal” indicating a position closer to the practitioner and “distal” indicating a position further from the practitioner. The terminology includes the above-listed words, derivatives thereof, and words of similar import.

Throughout the description and claims of this specification, the word “comprise” and variations of the word, such as “comprising” and “comprises”, means “including but not limited to”, and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention.

What is claimed is:

1. An interoperative blood loss quantification device comprising:
a housing;
a peristaltic pump positioned within the housing;
a stepper motor positioned within the housing and driving the peristaltic pump;
a cuvette positioned within the housing and configured to receive fluid moved by the peristaltic pump;
a spectrophotometer positioned within the housing and configured to provide an absorbance signal indicative of properties of the fluid flowing through the cuvette; and
one or more processing circuits comprising one or more memory devices coupled to one or more processors, the one or more memory devices configured to store instructions thereon that, when executed by the one or more processors, cause the one or more processors to:
control operation of the stepper motor,
determine a volume of fluid moved through the peristaltic pump,
determine a blood concentration based on the absorbance signal received from the spectrophotometer, and
determine a total volume of blood moved by the peristaltic pump based on the volume of fluid and the blood concentration.

2. The interoperative blood loss quantification device of claim 1, wherein the housing includes a vented body sized to contain the peristaltic pump, the stepper motor, the cuvette, the spectrophotometer, and the one or more processing circuits comprising one or more memory devices,
wherein the housing further includes a vented cap, a vented bottom bumper, and an operating room attachment bracket.

3. The interoperative blood loss quantification device of claim 1, wherein the housing includes a caddy removably received within the housing and supporting the peristaltic pump, the stepper motor, the cuvette, the spectrophotometer, and the one or more processing circuits comprising one or more memory devices.

4. The interoperative blood loss quantification device of claim 3, wherein the caddy includes a first caddy configured to support the cuvette and the spectrophotometer, and a second caddie configured to support the peristaltic pump and the stepper motor.

5. The interoperative blood loss quantification device of claim 1, further comprising a display coupled to the housing,
wherein the one or more memory devices are further configured to store instructions thereon that, when executed by the one or more processors, cause the one or more processors to generate a graphical user interface representing at least one of the volume of fluid moved through the peristaltic pump, the blood concentration, or the total volume of blood moved by the peristaltic pump.

6. The interoperative blood loss quantification device of claim 1, further comprising a communications interface configured to wirelessly transmit information from the interoperative blood loss quantification device including at least one of the volume of fluid moved through the peristaltic pump, the blood concentration, or the total volume of blood moved by the peristaltic pump.

7. The interoperative blood loss quantification device of claim 1, wherein the spectrophotometer includes a laser configured to obtain a dual isosbestic point measurement.

8. The interoperative blood loss quantification device of claim 1, wherein the spectrophotometer includes a first laser defining a first wavelength of about 520 nanometers, and a second laser defining a second wavelength of about 808 nanometers.

9. The interoperative blood loss quantification device of claim 1, wherein the cuvette includes a cover configured to inhibit light pollution of the cuvette during use.

10. An interoperative blood loss quantification system comprising:
a suction head;
a main reservoir fluidly coupled to the suction head;
a vacuum configured to provide a negative pressure within the main reservoir;
a blood loss quantification device including:
a peristaltic pump configured to move fluid from the main reservoir,
a stepper motor driving the peristaltic pump,
a cuvette configured to receive fluid moved by the peristaltic pump,
a spectrophotometer configured to provide an absorbance signal indicative of properties of the fluid flowing through the cuvette, and
one or more processing circuits comprising one or more memory devices coupled to one or more processors, the one or more memory devices configured to store instructions thereon that, when executed by the one or more processors, cause the one or more processors to:
control operation of the stepper motor,
determine a volume of fluid moved through the peristaltic pump, and
determine a total volume of blood moved by the peristaltic pump based on the volume of fluid and the absorbance signal; and
an end reservoir fluidly connected to the blood loss quantification device.

11. The interoperative blood loss quantification system of claim 10, further comprising a safety reservoir positioned fluidly between the main reservoir and the vacuum.

12. The interoperative blood loss quantification system of claim 10, wherein the one or more memory devices are further configured to store instructions thereon that, when executed by the one or more processors, cause the one or more processors to:
determine a blood concentration based on the absorbance signal, and
determine the total volume of blood moved by the peristaltic pump based on the volume of fluid and the blood concentration.

13. The interoperative blood loss quantification system of claim 10, wherein the blood loss quantification device further includes a housing, and
wherein the peristaltic pump, the stepper motor, the cuvette, the spectrophotometer, and the one or more processing circuits comprising one or more memory devices are received within the housing.

14. The interoperative blood loss quantification system of claim 10, wherein the blood loss quantification device further includes a housing and an operating room attachment bracket.

15. The interoperative blood loss quantification system of claim 10, further comprising a display,
wherein the one or more memory devices are further configured to store instructions thereon that, when executed by the one or more processors, cause the one or more processors to generate a graphical user interface representing the total volume of blood moved by the peristaltic pump.

16. The interoperative blood loss quantification system of claim 10, wherein the blood loss quantification device further includes a communications interface configured to wirelessly transmit information including at least one of the volume of fluid moved through the peristaltic pump, or the total volume of blood moved by the peristaltic pump.

17. The interoperative blood loss quantification system of claim 10, wherein the cuvette includes a cover configured to inhibit light pollution of the cuvette during use, and
wherein the spectrophotometer includes a first laser defining a first wavelength of about 520 nanometers, and a second laser defining a second wavelength of about 808 nanometers.

18. A method of interoperative blood loss quantification comprising:
moving a fluid from a main reservoir to a cuvette of a blood loss quantification device with a peristaltic pump;
determining a blood concentration of the fluid;
determining a total volume of fluid moved by the peristaltic pump; and
determining a total blood volume moved by the peristaltic pump based on the blood concentration and the total volume of fluid.

19. The method of claim 18, further comprising determining the total blood volume based on a hemoglobin concentration.

20. The method of claim 18, wherein determining the blood concentration includes determining an absorbance value of the fluid using a spectrophotometer including a first laser defining a first wavelength of about 520 nanometers, and a second laser defining a second wavelength of about 808 nanometers.
